(19) United States
(12) Patent Application Publication
(10) Pub. No.: US 2014/0228756 A1
(43) Pub. Date: Aug. 14, 2014

(54) INSTRUMENT FOR ASSISTING A USER DURING INJECTION, A METHOD OF USE AND A SYSTEM COMPRISING SAID INSTRUMENT

(71) Applicant: Jakob Dahl Thomsen, Holte (DK)

(72) Inventor: Jakob Dahl Thomsen, Holte (DK)

(73) Assignee: IN.TOOL APS, Holte (DK)

(21) Appl. No.: 14/342,106

(22) PCT Filed: Aug. 29, 2012
(86) PCT No.: PCT/DK2012/050321

§ 371 Date: Feb. 28, 2014

(30) Foreign Application Priority Data
Aug. 29, 2011 (DK) PA 2011 00648
Oct. 26, 2011 (DK) PA 2011 00815
Jun. 18, 2012 (DK) PA 2012 70339

(51) Int. Cl. A61M 5/31; A61M 5/42

(52) U.S. Cl. CPC A61M 5/3137; A61M 5/427; A61M 2005/3139; A61M 2205/587; A61M 2205/586
USPC 604/116; 604/227

(57) ABSTRACT

The present invention relates to an instrument (1) for assisting a user during injection of a substance by a delivery system (2), said instrument comprising: A grip unit (5) and means for attaching the grip unit to the delivery system. Said grip unit is arranged to be held by a first hand of the user while a second hand of the user controls the delivery system. The invention further relates to a method of use and a system comprising said instrument.

### FIELD OF INVENTION

The invention relates to a method and an instrument for use in delivery systems in connection with the user's own injection of medication. The method and the instrument are particularly suitable for self-subcutaneous injection of medication. The method and the instrument make it possible for the user to apply both hands for controlling an delivery system while using this system. Furthermore, illumination of the injection site independent of daylight or room light is achieved by a specific design/embodiment of the instrument.

No methods or instruments of the type described are known.

### BACKGROUND OF INVENTION

When using delivery systems in connection with injection of medicine yourself—without using an instrument of the type described here the user will have to apply both (his/her) hands independently of each other to the different tasks involved without being able to have the hands interact or cooperate. Especially in case of subcutaneous injection of medication, the user often wish to fold or raise the skin at the injection site with one of his/her hands (usually the left hand), while the second hand (usually the right hand) independently of the first hand, while keeping the skin raised or folded by the first hand, initially must inject the needle of the delivery system through the skin and then, (while the user maintains his/(her first hand on the skin and/or keeps the skin at the injection site folded or raised by the first hand) has to release the medication through the needle of the delivery system by applying pressure to the release button of the delivery system.

Without using the method described herein the second hand is holding and activating the delivery system without any support and vibrations generated in connection with the injection are transmitted to the needle in the skin. The vibrations occur partly because of the pressure of the fingers to the release button of the delivery system (as the thin needle does not meet any significant resistance under the skin), and partly due to the fact that the hand which carries out the injection primarily is controlled by the strong wrist and arm muscles which are not suitable for precise control of the small movements required for injection of medicine.

### SUMMARY OF INVENTION

Accordingly it is an object of the present invention to provide an instrument for assisting in delivering a medication from an injector to a body, where the use of the instrument overcomes the drawbacks of the above-mentioned complex manoeuvre.

According to a further aspect of the present invention is provided a method which helps eliminate vibrations and eliminate transfer of any generated vibrations from the delivery system to the skin e.g. during injection of insulin.

The above problems are reduced and the objects and aspects and other advantages are achieved by the invention by establishing a physical link between the user's two hands during use of the delivery system making it possible for both hands to take part in the injection and making it possible to use the body for support during the injection process. The method can be implemented by an instrument (an attachment and a holder). The very specific details of the instrument will depend on the delivery system used. The description of the instrument falls into two parts; however, the method and the instrument can be carried out with the grip unit alone if the delivery system in question is prepared for this option. If the delivery system is not designed for the method, this method can be implemented by an instrument, which both includes the attachment (grip unit) and a holder for the delivery system.

Many people, including those suffering from conditions such as diabetes, use some form of infusion therapy in order to maintain control of their diseases. To mention an example, a huge number of people use insulin pens (also called injectors) for daily insulin therapy through subcutaneous injection. This therapy often requires use of one hand to press together a skin fold and one hand to activate the injector (insulin pen). This maneuver is rather complex and requires a calm and controlled hand movement in order to be carried out conveniently. Especially the fact that the injector is controlled by only one hand is the origin of the problems and inconveniences, which the invention addresses.

Thus according to the present invention is provided an Instrument for assisting a user during injection of a substance by a delivery system, said instrument comprising:
A grip unit
Means for attaching the grip unit to the delivery system
   wherein said grip unit is arranged to be held by a first hand of the user while a second hand of the user controls the delivery system. This means that by using the present invention coordination of the two hands during injection is made easier. This is achieved in that the first hand holds the grip, and thereby supports the delivery device during the injection. Hereby, the instrument helps stabilize the delivery device during injection and thus helps keep the delivery device steady even during activation of the delivery device and during the time it takes for the injection to take place.

The grip unit may be any suitable unit adapted for providing a grip by using one finger or several fingers.

The length of the grip may be adapted to suit the intended use—e.g. the use of a user with a small hand or a large hand. Thus e.g. the grip may be between 3 and 8 cm or even between 2 and 12 cm to suit specific hand positions during use or even enable a grip suited for children.

Hereby, the instrument according to the invention makes is possible to assist and facilitate the calm and controlled hand movements that are required during subcutaneous injection. The effect from shaking hands during the delivering of a medication from an injector can be reduced by using the instrument according to the invention.

If the delivery system is an insulin injector the present invention can be applied with great advantages. The insulin injection takes time which requires that the user holds the delivery system steady for this time, which can be a straining or even impossible task depending on the physical ability of the user and also e.g. of the place of the injections site on the user body. When the instrument of the present invention is used, the user is using both hands—one hand holding the grip unit supported by the body and another hand activating the delivery device and holding it during the time it takes to perform the injection. This means that instead of having a passive first hand as is the case without the present invention, now the first hand is active in the process of supporting the delivery system.

The grip preferably extends away from the longitudinal direction of the delivery system thereby forming a grip which is easy to grasp for the user. The grip may for example extend in an angle between 70-100° e.g. close to perpendicular to the longitudinal direction of the delivery system.

The grip unit is preferably attached, either directly to the delivery system or via the holder, at the end of the delivery system near the needle.

Here insulin injectors are mentioned however the invention is suitable for all types of injectors.

Thus the instrument according to the invention may provide a simple and reliable instrument for assisting in self-injection for delivering a medication from an injector (delivery system) to a patient by the patient.

Preferably the means for attaching the grip to the delivery system comprises a holder for receiving the delivery system, i.e. preferably the grip is attached to the delivery system by a holder in which the delivery system can be inserted.

If the holder and the grip is a single unit, it is possible to achieve an instrument which is easy to handle and which provides an advantageous stability of the holder and grip relatively to each other.

Thus preferably the holder can either be permanently attached to the grip unit or be configured to be releasable attached to the grip unit.

The holder can be used to mechanically fixe different types of injectors (delivery systems) to the instrument so that the instrument can be applied for assisting in delivering a medication from different types of delivery systems having various shapes.

It is also possible that the holder and the grip are attached to each other by a hinge, joint or similar means allowing the grip and holder to be adjusted with respect to each other. Hinged embodiments of the present invention may allow the instrument to be folded for storage and/or provide the ability to select a specific angle between holder and grip. However, in hinged embodiments it is preferred that the hinge or related means are arranged to ensure stability of the grip and holder with respect to each other during use.

Also a hinge, joint or similar may provide a foldable instrument, thus providing a very compact instrument.

The coupling joint may be any suitable type of joint. The coupling joint may by way of example be a ball joint, a hinge joint or a flexible joint. It may be an advantage that an attachment member for mechanically fixing the grip unit to an injector is attached to the grip unit.

In some advantageous embodiments the holder is arranged to at least partly engage around the delivery system.

For example the holder may form at least in parts a spiral, or coil providing a structure into which the delivery system can be inserted and which supports the delivery system several at points along the length direction of the delivery system and which further provides a structure supporting the delivery system from all sides. Hereby, a flexible and reliable way of secure the injector to the holder can be achieved.

The spiral or coil member may comprise one of several coils and the coils may have different geometry, sizes and mechanical properties in order to meet different requirements (e.g. be adapted to fit to injectors having different thicknesses e.g. being tapered)

It may be beneficial that the holder comprises at least one support member configured to support the injector when the injector is secured to the holding member, where the at least one support member comprises:
a support element extending basically parallel to the longitudinal axis of the delivery device and
a bracket member extending transversely to the longitudinal axis of the delivery device so that the bracket member is configured to radially support an injector when the injector is secured to the holding member.

Hereby the support member can provide a reliable and secure support of the injector. Moreover, this type of support member may allow for adjustments of the radial position of the injector prior to a subcutaneous injection of e.g. insulin.

It may be an advantage that the bracket member is configured to match the outer shape of an injector. If the intended injector is circular cylindrical, it may be preferred that bracket member has a matching shape (an arced shape). However; other shapes of the bracket would be possible when the intended injector is circular cylindrical.

It may be an advantage that the holder comprises two support members being axially displaced from one another. The support members may have the same geometry, however; it is also possible to have support members of different size or shape (this may be an advantage if the injector is conical).

The holder may also form at least in part e.g. a crescent shape extending along at least a part of the longitudinal axis of the delivery system thereby providing a structure into which the delivery system may be slid or clicked and which similar to the spiral structure supports the delivery system over larger areas and which supports the delivery system from various angles.

Thus in several preferred embodiments the holder comprises retaining parts for receiving the delivery system and fastening the delivery system in a desired position in the holder.

Theses retaining parts may be arranged to grip at least partly around one or more parts of the delivery system and/or to engage with one or more corresponding parts on the delivery system.

The retaining parts may for example be at least partly circumferential parts e.g. crescent shaped parts griping at least partly around one or more parts of the injector i.e. delivery system.

Thereby the holder may both be used for attachment of the grip as well as keeping the grip and delivery system fixed relatively to each other.

Preferably the holder is arranged to ensure that the second hand grasp both the delivery system and at least part of the holder during activation of the delivery system thereby fixating holder and delivery system relatively to each other. This grasp of the second hand can e.g. be provided by the mentioned spiral and crescent structures which are at least partially open. The open structures means that when the user grabs around the delivery system engaged in the holder, the hand will hold both the holder and the delivery system and thereby provide further stability to the system.

In some embodiments the grip is permanently attached to the delivery system for example if the grip is cast together with a part of the delivery system. It is also a possibility that the grip can be attached permanently to the delivery system by the user after the user obtains the instrument.

In case the grip is attached permanently to the delivery system it may be an advantage that the means for attaching the grip to the delivery system comprises a flexible structure such as a joint allowing the grip unit to be twisted and/or folded and thereby e.g. aligned with the longitudinal direction of the delivery system to provide a compact grip+delivery system unit and thereby allow the user to use the grip by choice.

If the grip is releasably attached to the delivery system the grip may be attached to the delivery system when needed and may be detached e.g. when the delivery system is stored or transported. Also a releasable grip allows the user to use the instrument by choice, for example if an injection is to be made in a hard to reach part of the body.

In several advantageous embodiments the means for attaching the grip to the delivery system comprises a first lock part on the grip unit arranged to engage with a corresponding second lock part of the delivery system thereby allowing the user to attach the grip to the delivery system when desired. Preferably the first and second lock part together provides a releasable but yet stabile attachment of the grip to the delivery system.

If the delivery system is arranged with a second lock part, the grip unit may be attached directly to the delivery system without a holder.

Solutions with a first and second lock part will often require that the delivery system is manufactured to include the second lock part. However, it is also imagined that the second lock part can be fitted on the delivery system by the user after acquisition of a delivery system.

The grip is in preferred embodiments arranged to allow the user to at the same time hold the grip with the first hand while at the same time using the fingers of the first hand for example to create a skin fold at the injection site which may be an advantage e.g. during insulin injection. A grip which comprises an S shaped part or a 8 shaped part arranged to receive at least two fingers of the user allows the user to engage in a grasp of the grip in a simple movement while at the same time providing a stable grasp and a grasp which allows movement of the fingers. For example during insulin injections in may be a significant advantage that the user is able to use the fingers of the first hand to create a skin fold at the injection site.

The grip may have various shapes including a simple bar extending at a desired angle with respect to the delivery system, or it may have more complex shapes.

If the grip is curved to fit at least part of the curvature along the length direction of the delivery system when not in use the grip can easily be stored together with the delivery system. For example the grip which fits the curvature of the delivery system, can be attached to the delivery system by a clip and thereby be stored and transported together with the delivery system without catching cloths or objects for example in a hand bag.

In advantageous embodiments the grip is curved to fit under the clips of the protective cap of a delivery system when not in use. It is also possible that other parts of the delivery system are adapted to receive the grip when not in use. For example the protective cap, or other part of the delivery system, may have one or more recesses for receiving the grip in order to have a compact design with as few protruding parts as possible thereby avoiding entanglement during storage and transport.

If the grip is a basically flat structure, e.g. a plane S shape there is no preferred direction of use. However, if the grip is curved there may be a preferred direction of use which may be relevant depending on people using their left or right hand as the first hand. However if the grip comprises a first lock part at each end of the grip the direction of attachment to the delivery system may be chosen by the user for example to fit a preferred first hand.

If the user receives two differently curved or shaped grips (e.g. each curved in the opposite direction compared to the other grip) the user may choose the preferred grip under different circumstances and/or needs.

If the grip unit comprise a first and second grip part, the first and second grip part may be adjustable with respect to each other in order in order for a user to optimize the grip. This adjustability may be achieved e.g. by a joint between the first and second grip part or the grip may comprise one or more sections with a flexible material.

The body may be the body of a human being or an animal. The instrument can be used by health care personal or veterinary staff when applying medication delivery devices to patients or animals.

It may be an advantage that the instrument comprises an illuminator. Hereby the instrument can be used in the absence of the required amount of light.

The illuminator may comprise a light source selected to ensure that relevant features at and/or near the injection site is clearly seen. For example the wave length of the light can be chosen for blood vessels to stand out clearly.

The geometry of the illuminator with respect to the injection site may be chosen so that the illuminator can be used to search the skin for an optimal injection site as well as give a sufficient illumination of the injection site before and during injection.

The instrument may be made in any suitable material. The grip unit and the holding may, for instance be made in metal (e.g. steel) or plastic or fiber reinforced plastic.

The grip unit and the holding may for example be made by a cord or wire material.

The grip and/or holder may also be made at least partly of a moldable material which enables shaping the holder to fit a specific delivery system and/or the grip to be adapted to the preferences of the user.

Thus according to the present invention the instrument may consist of a grip unit arranged to be directly attached to the delivery system either permanently or releasable or the instrument may consist of a grip unit attached permanently or releasable to a holder into which the delivery system is inserted for use.

Generally it is advantageous if the grip is attached to the delivery system, whether it is by the holder or a lock system, in a way which ensures stability of the attachments when pressure is applied during use. For example, a lock system can be angled in a manner where the first and second lock parts are pressed further into the lock position when the use pressure is applied. Similarly a holder can be provided with indentations, protrusions, conical parts etc. preventing that the delivery system is pressed out of the engagement with the holder during use.

A system comprising an instrument as described above and a delivery system, wherein the instrument comprises:
A grip unit
Means for attaching the grip unit to the delivery system
   and wherein the instrument comprises means for receiving the grip unit at least during use of the delivery system. In several advantageous embodiments the instrument and delivery system is provided as a system. In these embodiments it is especially advantageous if the delivery system is arranged to receive and/or at least partly match the shape of the grip unit and vice versa.

For example the delivery system may be arranged to receive and store the grip unit while not in use in order to provide a simple and portable system which prevents that the grip unit gets lost.

A system may be provided with different grip units enabling the user to choose the most suitable grip depending on e.g. hands size, preferred main hand, place of injection etc.

The present invention further relates to a method for establishing a link and cooperation between both of the user's hands while applying medical delivery systems in connection with self-injection of medication, comprising the steps of:
establishing a physical connection between the user's two hands by means of an instrument comprising a grip, which can be controlled by the fingers on a first hand, said grip being attached to the delivery system
by the first hand holding the grip while resting the first hand against the body of the user
by the second hand grasping and activating the delivery system
thereby ensuring increased control and/or stability during injection

Preferably the instrument used in the method is the method according to the present invention.

### DESCRIPTION OF DRAWINGS

FIG. 1 shows the instrument in use with an delivery system

FIG. 2 shows an embodiment of a grip unit

FIG. 3 shows an embodiment of an alternative grip unit

FIG. 4 shows a first embodiment of an instrument according to the present invention

FIG. 5 shows a second embodiment of an instrument according to the present invention

FIG. 6 shows a third embodiment of an instrument according to the present invention

FIG. 7 shows a fourth embodiment of an instrument according to the present invention

FIG. 8 shows a grip unit stored with a delivery system

FIG. 9a shows the grip unit of FIG. 8

FIG. 9b shows the grip unit from FIGS. 8 and 9b attached to a delivery system

FIGS. 10a and 10b shows further embodiments of grip units

FIG. 11 shows a first and second lock part in form of a bar and bore

FIG. 12 shows a fifth embodiment of an instrument according to the present invention

FIG. 13 shows an embodiment of an instrument according to the present invention including a light source

In the following the invention will be further described with reference to the drawings. The drawings are provided for detailed description and examples and are not to be construed as limiting to the invention.

### DETAILED DESCRIPTION OF THE INVENTION

The instrument according to the present invention may be adapted to the different delivery systems and adapted to suit various situations as will be clear from the following sections. I.e. depending of the delivery system different embodiments of the present invention can be used, or one embodiment may be adjusted to e.g. work with a certain type of delivery system.

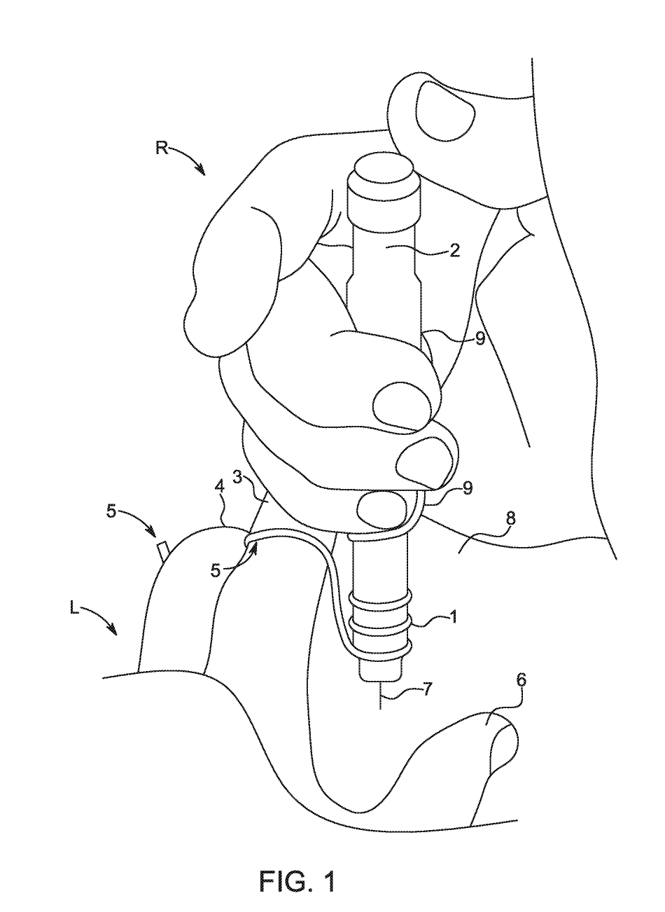

FIG. 1 illustrates an instrument 1 according to the invention being used for delivering a medication from an injector 2 to a patient, by the patient.

The patient uses both the left hand L (here first hand) and the right hand R (here second hand) while applying the instrument 1. The patient uses the left hand L index finger 3 as well as the long finger 4 to engage the grip unit 5. The left hand L rest against the body and its ring finger (not shown) and left hand L thumb 6 can be used to hold the skin or to gather a skin fold so that the needle 7 of the injector 2 can be inserted into and fixed to the receiving body 8 the best possible way e.g. through subcutaneous injection.

The patient holds the injector 2 in the right hand R and the injector 2 is mechanically fixed to the holding member 9 that is mechanically connected to the grip unit 5. Therefore, the movement of the injector 2, relatively to the injection site, is restricted due to the constrains caused by the fixation by the holding member 9 and the grip unit 5. Accordingly, the instrument 1 reduces the influence of unintended movements (e.g. a shaking hand) on the process of delivering a medication from the injector 2 to the patient.

The instrument 1 is for example suitable for assisting self-injection by a injection system of delivering a medication from an injector to a patient, by the patient.

Is should be underlined that FIG. 1 shows an example of a right-handed user of the delivery system 2. Some embodiments of the instrument 1 can be used by both right-handed and left-handed persons. Some embodiments may be suited for bi dextral use and some embodiments may be found in a right and/or left handed version.

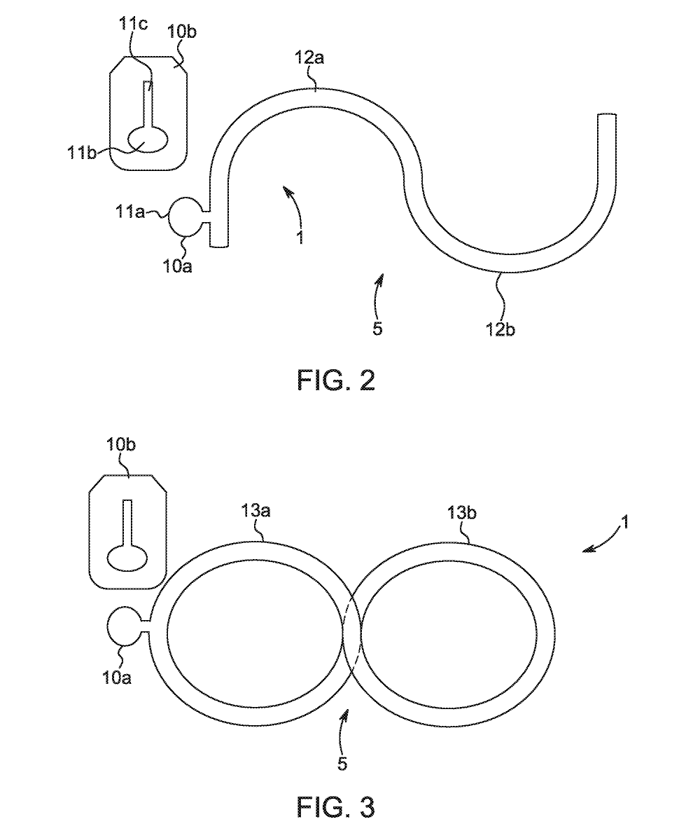

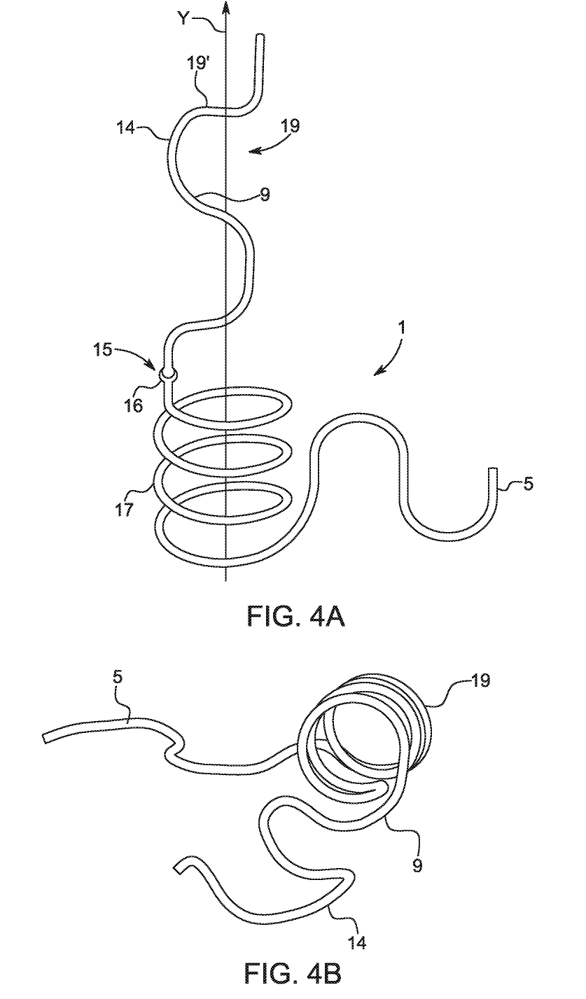

FIG. 2 and FIG. 3 show two different basic versions of an instrument which consists of a grip unit (directly connected to the delivery system as an attachment).

The design of the grip unit 5 can take a form which can be described as a reversed lying “s” (FIG. 2) or be similar to a lying 8-digit (FIG. 3). As mentioned above, the grip unit 5 is to be attached as an attachment to the lower part of the delivery system 9 if that system is prepared for this option by means of a coupling mechanism. There are different coupling mechanisms available and the example shown in FIG. 2 and FIGS. 3 10a and 10b is given for illustrative purposes.

The grip in FIG. 2 comprises a first grip part 12a closest to the delivery system (when in use) and a second grip part 12b further away from the delivery system (when in use). In the shown embodiment the first grip part 12a is arranged to receive the index finger of the first hand of the user. The second grip part 12b is arranged to receive one or two of the middle fingers of the user during use.

Similarly FIG. 3 shows a grip unit 5 comprising a first 13a and second 13b grip part.

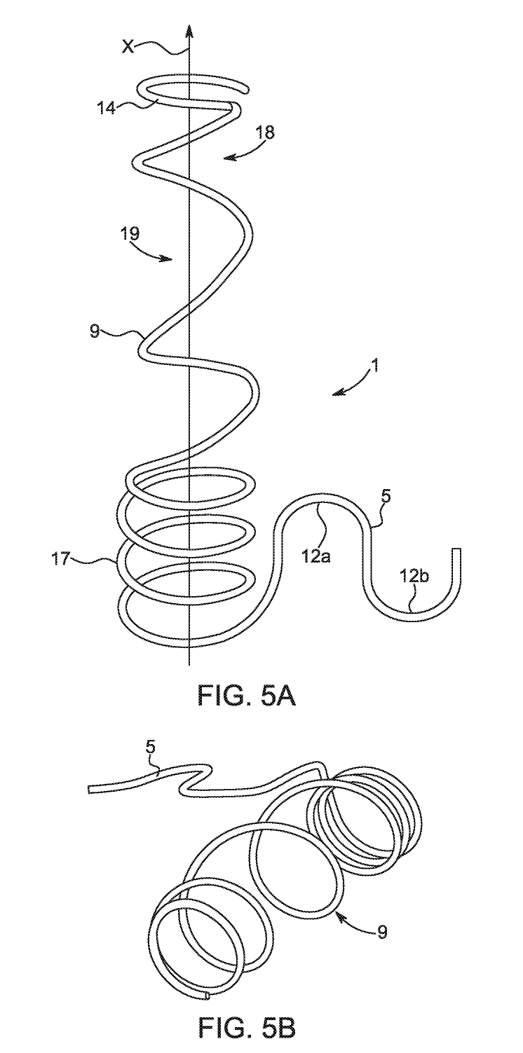

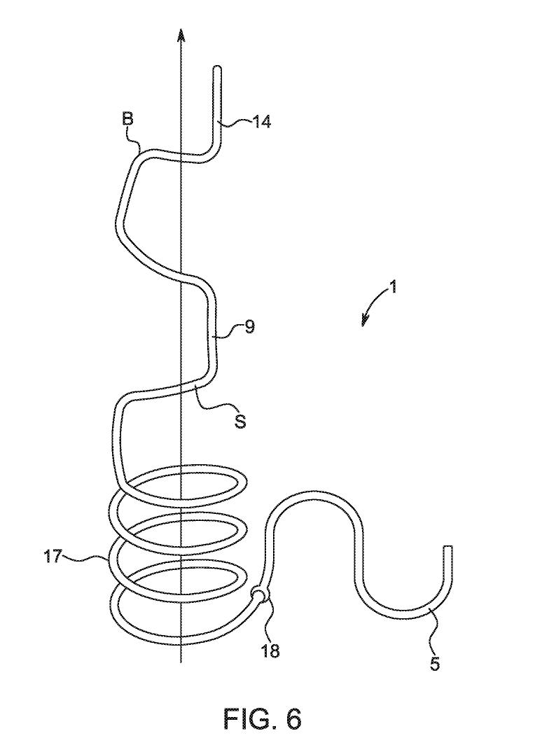

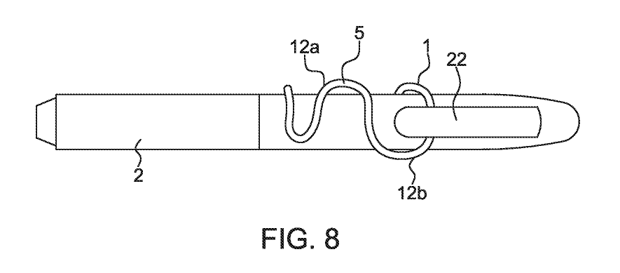

If the instrument includes both a grip unit 5 and holder 9 (as mentioned to be used in case the delivery system is not prepared for coupling with grip unit 5), then the grip unit 5 will be an attachment to the holder 9 which will function as a holder for a common delivery system. The coupling of the grip unit 5 (attachment) to the holder can be achieved either by a stiff link (as shown in FIGS. 4 and 5) or be flexible (illustrated by FIG. 7). The grip unit 5 and the holder 9 combined will ensure the connection and interaction between the user's two hands during the use of the delivery system.

Concerning the design of the grip unit 5, it has to be noted that its upward curve shown in FIG. 1 preferably is closest to either the delivery system 2 in use or the holder 9. The grip unit 5 may be designed in a way that will allow two of the user's fingers—usually the index and middle fingers—to control this part of the instrument, thus, the dimensions of the arcs or circles in the attachment are preferably made in sizes giving room to the index and middle fingers of the user. The arcs and circles of the grip unit 5 can be constructed with thicknesses of approximately 1 mm however; a thickness 2, 3 or more mm may provide a softer grip. Also the thickness may be non-uniform over the length of grip and/or holder.

If the delivery system 2 is prepared for this option, the grip unit 5 can be used directly together with the delivery system 2 without the need for a holder 9. This is achieved by a coupling mechanism on both the grip unit 5 and the delivery system 2 as shown by 11a and 11b in FIG. 2 and FIG. 3. The coupling mechanism can take the form of a little knot 11a on the grip unit 5, which can be introduced into a hole 11b and pushed into a groove 11c on the delivery system 2. In this way grip unit 5 can be flexibly located in relation to the delivery system 2 e.g. rotatably in the coupling mechanism.

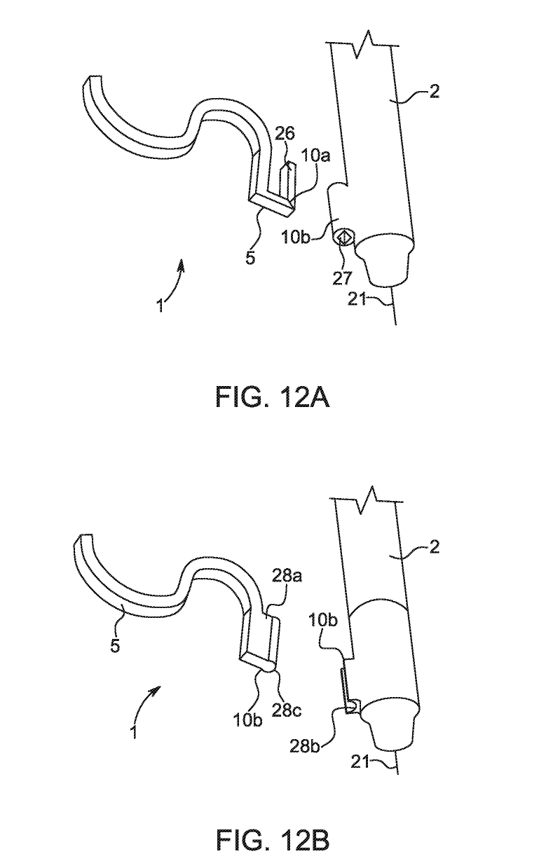

Other alternative coupling mechanisms are shown in FIGS. 12a and 12b.

FIGS. 4a, 4b and FIGS. 5a, 5b show two different basic versions of an instrument 1 which may be used with delivery systems (not shown) not readily prepared for use with the grip unit 5.

FIGS. 4a and 4b show a partly circular (curved) version of the upper part 14 of the holder 9 for the delivery system (not shown), while FIGS. 5a and 5b show a fully closed circular form (stretched spiral) of this upper section of the holder 14. The connection between the grip unit 5 and the holder 9 can be either flexible by a hinge or similar as will be discussed later.

A flexible coupling between upper 14 and lower 17 holder part as shown in FIG. 4 can be achieved in several ways. The flexible coupling mechanism shown in FIG. 4 consists of a curved platform 16 which receives and retains the upper part of the holder 14. Hereby the upper part 14 may be rotatable in relation to the curved platform 16 and thus with respect to lower part 17 of the holder and grip unit 5.

Some delivery systems are circular with more than one diameter of the system (but can, in principle, also be angular). If the top diameter of the delivery system is smaller than any of its downstream diameters, an open form of the upper holder part 17 of the instrument as shown in FIGS. 4a/4b may be the best one. This is due to the fact that it will be difficult—sometimes impossible—to introduce a delivery system with the a non-uniform diameter into an instrument designed with a uniform diameter as shown in FIGS. 5a/5b if the holder 9 is to be enclosing the delivery system relatively closely (whereby the best result may be obtained).

Both the open form of the upper part of the holder and the flexibility of the upper and lower part of the holder with respect to each other as indicated in FIG. 4a may facilitate easy insertion and a stable hold of an delivery system with a non-uniform diameter.

It is to be noted that most delivery systems 2 are circular, meaning that the holder 9 consisting of upper 14 and lower 17 part in most cases also will have to be a circular, possibly open and curved.

The lower part 17 of the holder 9 consists of a circular or angular form adapted to the lower part of the delivery system used with the instrument in order for the lower part of the holder 17 to enclose the lower part of the delivery system (not shown) relatively closely.

The shape and size of the upper part 14 of the holder 9 is adapted to the shape and size of the upper part of the delivery system so that this can be placed exactly in the instrument.

FIG. 4b shows a rotated view of the instrument of FIG. 4a.

Delivery systems which are circular with only one diameter, may readily be used together with an instrument designed as a stretched spiral or a tubular unit as shown in FIGS. 5a and 5b.

In any case, the size and shape of the instrument 1 may be adapted to the delivery systems depending on their specific shape.

If the upper part 14 of the holder 5 is constructed as a tubular unit, that part of the instrument must preferably have at least finger-thick openings 19 or holes on its sides to make it possible for the fingers of the hand of the user to be in contact with the delivery system 2 inside the instrument when the user's hand surrounds the instrument 1. This contact between fingers and delivery system 2 may be achieved with e.g. a stretched spiral design in the example shown in FIGS. 5a/5b, which allows sufficient room for the fingers of the user to enter into the openings. This way the user performing the injection can hold the delivery system 2 and the instrument 1 in a fixed position with respect to each other.

The thickness of the sides of the holder 9 will normally correspond to the thickness of grip unit 5 (e.g. 1 mm). The height of the holder 9 must preferably be adapted to the lower part of the delivery system to which the needle is attached. In order to avoid impediments to the access to the delivery system's release mechanism, which normally is located on top of the delivery system, the total length of the instrument 2 (corresponding to the length of holder 9) can be adapted to the specific delivery system.

FIG. 5b shows a rotated view of the instrument known from FIG. 5a.

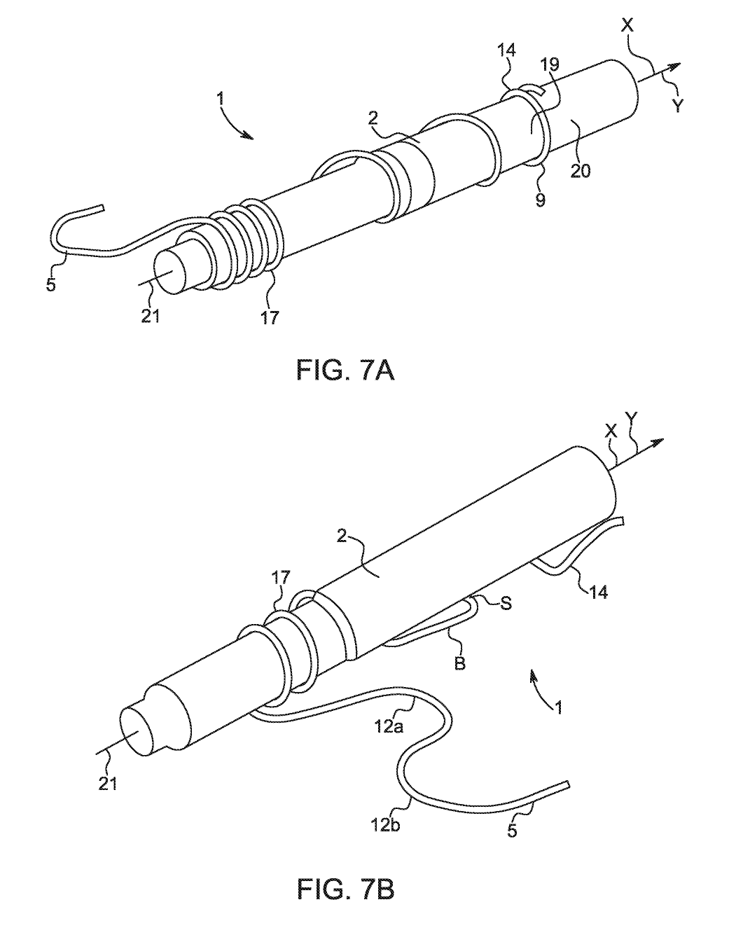

If the instrument 1 is designed with both a grip unit 5 and a holder 9, the coupling between these two units can either be stiff as in FIGS. 4 and 5 or be flexible as indicated in FIG. 6.

FIG. 6. Shows an instrument 1 comprising a grip unit 5 permanently but flexibly connected to the holder 9 by a joint 18 e.g. a ball joint. This joint 18 may provide a possibility of adjusting the angle between holder 9 and grip 5. The joint may also be arranged so that the grip unit can be twisted and folded with respect to the holder and thereby made to rest against the holder e.g. to minimize entanglement during storage or transportation.

FIG. 7a illustrates an injector 2 fixed to an instrument 1 according to the invention. The injector (delivery system) 2 has an elongate cylindrical body 20 and a needle 21 and the instrument 1 comprises an upper holder part 14 and a lower holder part 17 shaped to match the shape of the injector (i.e. delivery system) 2. The injector 2 is arranged in such a way that the longitudinal axis Y of the instrument 1 corresponds to the longitudinal axis X of the injector 2. The coil members 14, 17 are configured to prevent and/or reduce the radial motion of the injector 2 during injection.

FIG. 7b shows an injector 2 fixed to another instrument 1 according to the invention.

The instrument 1 comprises a grip unit 5 as described in the previous sections. The grip unit 5 has a first grip part 12a and a second grip part 12b. The instrument 1 has a holding member 9 comprising a coil around the injector 2.

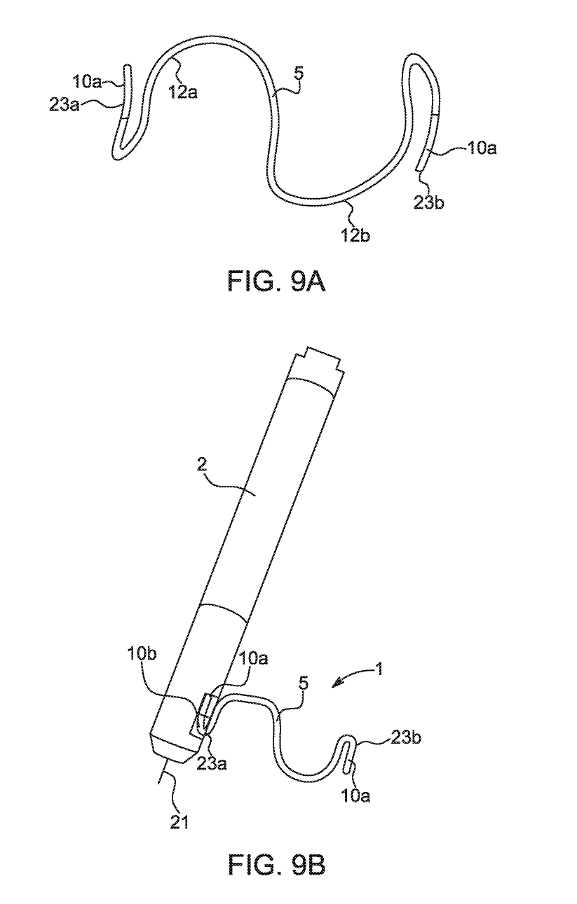

FIG. 8 shows an embodiment of the invention in which the grip unit 5 is arranged to be attached to the delivery system via a first and second lock part i.e. attachment means (not shown) on delivery system 2 and grip unit 5. In this embodiment the grip unit 5 is curved to match the curvature of the delivery system 2, thus enabling storage of the grip unit together with the delivery system when not in use. In the shown embodiment the grip unit 5 is arranged to fit under the clips 22 of the delivery unit 2 when not in use.

Preferably, the clip is part of the protective cap and where the grip is shaped to fit under the clips and to follow the curvature of the protective cap.

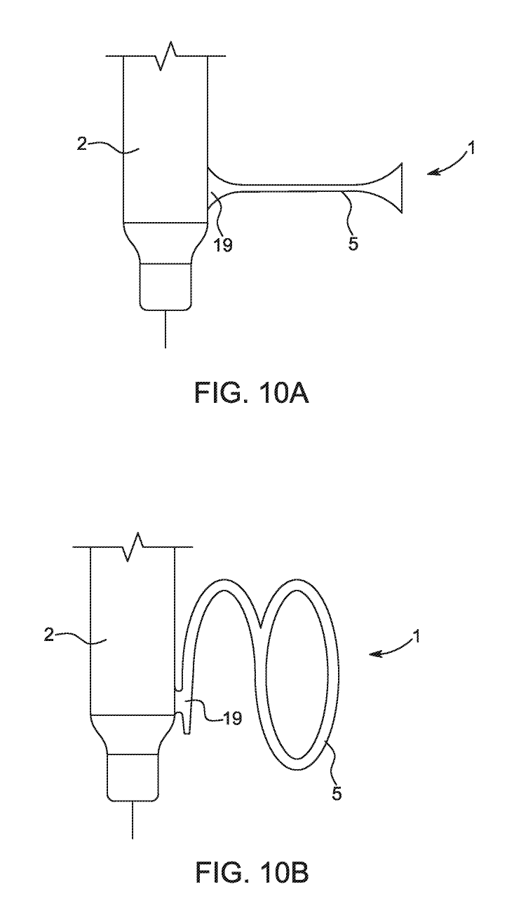

FIG. 9a shows the grip unit of FIG. 8. The grip unit 5 comprises a first 12a and second 12b grip part and a first 23a and a second 23b grip end, each end having first lock part 10a for attaching the grip unit 5 to the delivery system (not shown). This is an advantage as the curved shape of the grip unit 5 (as the grip unit is shaped to follow the curvature if the delivery system as shown in FIG. 8) in this embodiment may induce a preferred direction of use, but the two first lock 10a parts allows the user to choose the direction of attachment thus ensuring that the grip unit can be used by both left and right handed users

FIG. 9b shows the grip unit 5 and delivery system 2 known from FIGS. 8 and 9a coupled to each other by first lock part 10a and second lock part 10b arranged on grip unit 5 and delivery system 2 respectively.

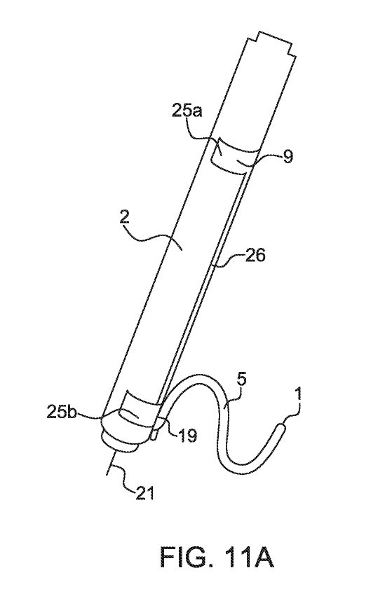

FIGS. 10a and 10b, show two alternative embodiments of a grip unit 5. FIG. 10a shows a grip unit 5 which extends perpendicular or substantially perpendicular to the delivery system 2 to which it is attached by attachment means 19. The shape of the grip unit 5 is basically a slightly curved bar. This embodiment offers a very simple solution to which comfort and/or improved grip may be provided by adjusting the curvature.

The attachment means 19 can comprise a first and second lock part and may either attached the grip unit 5 releasable to the delivery system or the attachment means 19 may form a permanent attachment between grip unit 5 and delivery system 2. In either case the attachment means may form a rigid coupling fixing the grip unit 5 and delivery system 2 with respect to each other. Alternatively the attachment means may form an at least partly flexible attachment making it possible to adjust e.g. the angle between delivery system 2 and grip unit 5.

FIG. 10b shows an embodiment of a grip unit which is a combination of the grip units known from FIGS. 2 and 3. I.e. it comprises a semicircular first grip part 24a and a circular second grip part 24b. The grip unit of FIG. 10b is attached to the delivery system 2 by attachment means 19 as described above.

The grip units of FIGS. 10 and 10b may be attached directly to the injector 2 or may releasable or permanently attached to a holder 9.

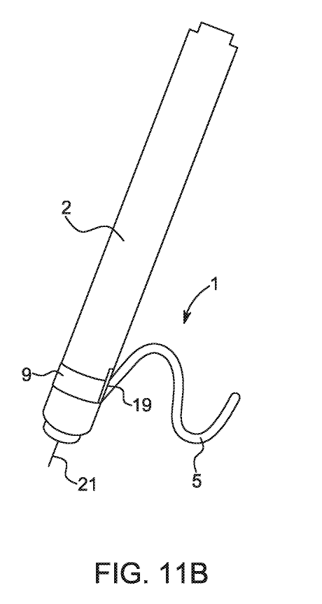

FIG. 11a shows and alternative embodiment of the holder 9 of the instrument 1. The holder 9 comprises an upper 25a and a lower 25b crescent shaped element partly encircling the delivery system 2 connected by a bar 26. The grip unit 5 is attached at or near the lower crescent shaped element 25b. The attachment of the grip unit 5 may be permanent or releasable. If the crescent shaped elements and the bar is made of e.g. thin materials the holder may add very little weight to the delivery system 2 as well as it may be arranged to fit smoothly to the delivery system 2. This may make it possible to keep the holder 9 attached to the delivery system 2 after use if desired and detached the grip unit 5 alone after use to store separately or together with the delivery system 2.

FIG. 11 b shows yet another embodiment of a holder according to the present invention. Here the holder simply forms a tight band around a part of the delivery system 2 near the needle. The grip unit 5 may be permanently or releasable attached to the holder.

As above the grip unit 5 of FIGS. 11a and 11 b is attached to the holder by attachment means 19 which are further discussed above under FIG. 10a.

FIG. 12a shows an alternative embodiment of the first 10a and second 10b lock part. In this case the first lock part is a bar 26 which is arranged to engage in the second lock part in form of a bore 27 on the delivery system (or alternatively on a holder). The bore 27 is located in a part contained in the delivery system.

The bar 26 and bore 27 may be shaped to match each other in different ways in order to allow fixation of the grip unit 5 at one or more angels with respect to the delivery system 2. In the present example the bore and bar have a square cross section but may have various other cross sections such as e.g. an octagon which will allow the grip and delivery system to be fixed with respect to each other in various angles.

FIG. 12b shows yet another embodiment of attachment means comprising a first 10a and second 10b lock part, In this embodiment the first lock part of the grip unit 5 is a semicircular bar 28a arranged to slide into second lock part 10b of the delivery system in for of a groove 28b. The semicircular bar 28a may have a collar 28c which prevents the first lock part 10a, 28a of sliding too far into the second lock part 10b i.e. groove 28b. In the present embodiment the first and second lock part are arranged to form a tight grip which is stabile but may provide the option of correcting the angle between delivery system 2 and grip unit 5 if desired without compromising the stability of the instrument during use.

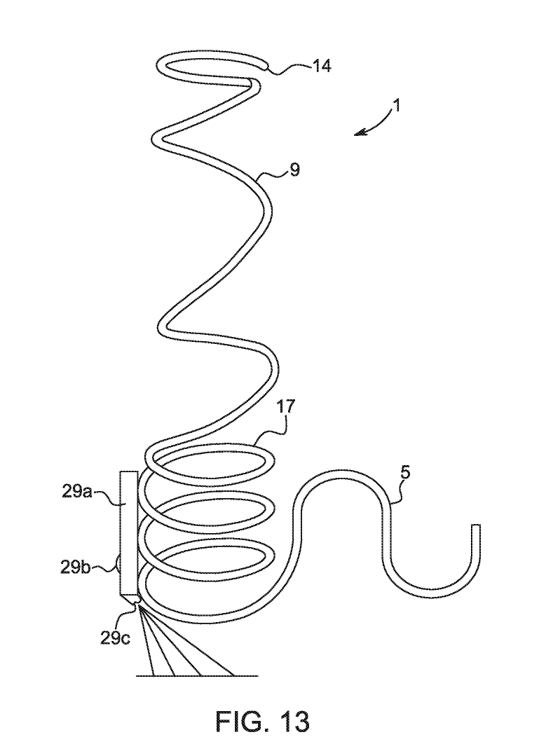

FIG. 13 shows a further embodiment of an delivery device 2 according to the invention. The delivery device 2 corresponds to the one shown in FIG. 1. The instrument 2 has a grip unit and a holding member 9 having a first coil member 14 and a second coil member 17. The instrument 1 comprises an illuminator 29 configured to illuminate the skin area into which the injector is intended to be inserted. Thus, the instrument 1 can be used in the absence of the required amount of light.

In use illumination of the injection site is normally ensured by daylight or room light without using any help. Shadows from the user's hands or other parts of the body will often impede a clear view of the injection site. These problems are solved by the placement of a battery-powered lighting unit on holder 9. FIG. 13 shows an example of a lamp 29a, switch button (29b) and light bulb (29c). Hereby, illumination of the injection site is improved.

The described invention FIG. 1-FIG. 13 creates the means for interaction—cooperation—between the user's hands and for support of the injecting hand during manual injection of medication, which improves control of injections and reduces vibrations during the injections. This can be described in more detail in this way:

If the grip unit 5 is used directly with the delivery system 2, the first step will be to connect the attachment to the delivery system by means of the coupling 10a 10b and to prepare the delivery system 1 for injection. Then the index finger and the middle finger of the hand, which may rest on the body and which can lift or fold the skin at the injection site, are inserted in grip unit 5 (e.g. the reversed lying “s” or Figure 8 formation), and the skin can be lifted or folded with the thumb and ring finger (or thumb and little finger) of the same hand. The needle 21 is introduced under the skin using the hand grabbed on the delivery system 2 in cooperation with the index finger and middle finger of the first hand and finally the medicine is released (with the thumb). The optional flexibility of both the coupling mechanism 10a 10b and the index and middle fingers on the first hand means that the positioning of the needle of the delivery system is flexible.

The functioning of an instrument 1 consisting of both a holder 9 and a grip unit 5 is in principle the same as described above. The first step is to prepare the delivery system 2 for injection and to insert the system into the instrument 1. Then the index finger and the middle finger of the hand, which is to test against the body of the user and which can lift or fold the skin at the injection site, are inserted in grip unit 5 and the skin is lifted or folded with the thumb and ring finger/little finger of the first hand. The second hand grabs holder 9 of the instrument 1 and thereby also the delivery system 2 which is inserted in the instrument 1 and introduces the needle 21 under the skin in cooperation with the index finger and middle finger of the first hand and finally releases the medication. The flexibility of the index and middle finger, possibly supplemented by a flexible coupling mechanism 18 means that the needle can be positioned in a flexible way. If the upper part of the holder 14 is designed similar to a stretched spiral, or e.g. is an entirely circular, tubular unit with finger thick holes or openings, the openings on upper holder part 14 implies that the fingers of the hand that is holding the holder 9 will also ensure securing of the delivery system 2 inserted into the instrument 1. Thus, the instrument 1 and the delivery system 2 are linked to each other (this is achieved in any case with an open version of 2b as shown in FIGS. 4a/4b).

The interaction between the two hands during the injection is more specifically obtained by two circumstances. On the first hand, the user's two fingers (index and middle fingers) will contribute to the control of the delivery system 2 during the different phases of the injection through the attachment grip unit 5. The second hand grabbed on the holder 9 and in contact with the delivery system 2 inserted into the instrument 1 will also influence the processes. This ensures more stability—even when the injection is terminated with pressure on the release mechanism of the delivery system.

### Items

1. A method for establishing a link and cooperation between both of the user's hands while applying medical delivery systems in connection with self-injection of medication, characterized by establishing a physical connection between the user's two hands by means of an instrument consisting of an attachment (grip unit, Unit 1), which can be controlled by the fingers of one of the hands, and which either can be connected directly to an delivery system with a coupling mechanism or be made together with an open holder (holder, Unit 2), the shape and size of which is adapted to the different delivery systems which can be controlled by the other hand and its fingers.
2. An instrument for carrying out item 1 for use in delivery systems in connection with the user's self-injection of medicine, characterized by an attachment (Unit 1) similar to a reverted lying “s” with curves (FIG. 2), which in size is adapted to different finger thicknesses and made with a thickness of the material of approx. 1 mm, which is connected to the delivery systems through a flexible coupling mechanism with the upward curve of Unit 1 closest to the delivery system.
3. An instrument for carrying out item 1 for use in delivery systems in connection with the user's self-injection of medicine, characterized by an attachment (Unit 1) designed as a lying 8-digit (FIG. 3) with circles, which in size can be adapted to different finger thicknesses and made with a thickness of the material of approx. 1 mm, which is connected to the delivery systems through a flexible coupling mechanism.
4. An instrument according to item 2 or 3 characterized in that a flexible coupling mechanism) is connected to the lower part of a holder (Unit 2), which is designed and shaped so as to adapt it to different delivery systems allowing the different delivery systems to be inserted into the holder which has at least finger-thick holes or openings on the sides if this upper part of the holder (Unit 2) is completely tubular or spiral shaped.
5. A method and an instrument according to item 2 or 3 characterized in that it by means of a fixed connection continues to the lower part of a holder (Unit 2), which is designed and shaped so as to adapt it to different delivery systems allowing the different delivery systems to be inserted into the instrument which has at least finger-thick holes or openings on the sides if this upper part of the holder (Unit 2) is completely tubular or spiral shaped.
6. A method and an instrument according to items 4 and 5 characterized in that a lightning unit with switch button and light bulb is placed on Unit 2 (FIG. 13).
A. An instrument for delivering a medication to a body from an injector characterised in that the instrument comprises:
a grip unit having at least one grip member configured to be held by a finger and
means for mechanically fixing the grip unit to the injector.
   B. An instrument according to item A characterised in that the grip unit comprises a first basically semicircular grip member connected to a second basically semicircular grip member, where the open end of the first grip member is directed in the opposite direction than the open end of the second grip member.
   C. An instrument according to item A characterised in that the grip unit comprises a first ring-shaped grip member mechanically connected to a second ring-shaped grip member.
   D. An instrument according to item A characterised in that the instrument comprises a holding member either being attached to the grip unit or being configured to be attached to the grip unit.
   E. instrument according to item D characterised in that the holding member comprises a coil member adapted to receive and secure an injector to the holding member.
   F. instrument according to item D or item E characterised in that the holding member comprises at least one support member configured to support the injector when the injector is secured to the holding member, where the at least one support member comprises:
a support element extending basically parallel to the longitudinal axis (X) of the instrument and
   a bracket member (B) extending transversely to the longitudinal axis (Y) of the instrument) so that the bracket member is configured to radially support an injector when the injector is secured to the holding member).
   G. An instrument according to item F characterised in that the holding member comprises two support members being axially displaced from one another.
   H. instrument according to item D-F characterised in that a coupling joint is arranged:
between a first part of the holding member and a second part of the holding member or
between the holding member and the grip unit.
   I. An instrument according to one of the item claim A-C characterised in that an attachment member for mechanically fixing the grip unit to an injector is attached to the grip unit.
   J. instrument according to one of the preceding items characterised in that the instrument comprises an illuminator.

1. An instrument for assisting a user during injection of a substance by a delivery system, said instrument comprising:
a grip unit, and
means for attaching the grip unit to the delivery system,
wherein said grip unit is arranged to be held by a first hand of the user while a second hand of the user controls the delivery system.

2. An instrument according to claim 1 wherein the delivery system is a medical injector.

3. An instrument according to claim 1, wherein the means for attaching the grip to the delivery system comprises a holder for receiving the delivery system.

4. An instrument according to claim 3, wherein the holder and the grip is a single unit.

5. An instrument according to claim 3, wherein the holder and the grip are attached to each other by a hinge.

6. An instrument according to claim 3, wherein the holder is arranged to at least partly engage around the delivery system.

7. An instrument according to claim 3, wherein the holder forms at least in parts a spiral or crescent shape extending along at least a part of the longitudinal axis of the delivery system.

8. An instrument according to claim 3, wherein the holder is arranged to ensure that the second hand grasps both the delivery system and at least part of the holder during activation of the delivery system thereby fixating the holder and the delivery system relative to each other.

9. An instrument according to claim 1, wherein the grip unit is releasably attached to the delivery system.

10. An instrument according to claim 1, wherein the grip unit is permanently attached to the delivery system.

11. An instrument according to claim 1, wherein the means for attaching the grip to the delivery system comprises a first lock part on the grip unit arranged to engage with a corresponding second lock part on the delivery system.

12. An instrument according to claim 1, wherein the grip unit comprises an S-shaped part or an 8-shaped part arranged to receive at least two fingers of the user.

13. An instrument according to claim 1, wherein the grip unit comprises a first lock part at each end of the grip unit.

14. An instrument according to claim 1, wherein the grip unit is curved to fit at least part of the curvature along the length direction of the delivery system when the grip is not in use.

15. An instrument according to claim 1, further comprising a light source.

16. A system comprising an instrument and a delivery system according to claim 1, wherein the instrument comprises:
a grip unit; and
means for attaching the grip unit to the delivery system;
wherein the instrument comprises means for receiving the grip unit at least during use of the delivery system.

17. A system according to claim 16, wherein the delivery system is arranged to receive and store the grip unit while not in use.

18. A method for establishing a link and cooperation between both of a user's hands while applying medical delivery systems in connection with self-injection of medication, comprising the steps of:
establishing a physical connection between the user's two hands by means of an instrument comprising a grip, said grip being attached to the delivery system, and
controlling the grip by the fingers of a first hand,
by the first hand holding the grip while resting the first hand against the body of the user,
by the second hand grasping and activating the delivery system,
thereby ensuring enhanced control and/or stability during injection.

19. The method according to claim 18 wherein the instrument is an instrument for assisting a user during injection of a substance by a delivery system, said instrument comprising:
a grip unit, and
means for attaching the grip unit to the delivery system,
wherein said grip unit is arranged to be held by a first hand of the user while a second hand of the user controls the delivery system.
